(19) United States
(12) Patent Application Publication
(10) Pub. No.: US 2024/0096449 A1
(43) Pub. Date: Mar. 21, 2024

(54) COMPUTE SCHEDULING FOR SEQUENCING ANALYSIS

(71) Applicant: ILLUMINA, INC., San Diego, CA (US)

(72) Inventors: Paul Smith, San Diego, CA (US); Bo Lu, San Diego, CA (US); Michael J. Carney, San Diego, CA (US); Hsu-Lin Tsao, Poway, CA (US); Gavin Derek Parnaby, Laguna Niguel, CA (US); Mohamed Amine Bergach, San Diego, CA (US)

(73) Assignee: ILLUMINA, INC., San Diego, CA (US)

(21) Appl. No.: 18/243,594

(22) Filed: Sep. 7, 2023

Related U.S. Application Data
(60) Provisional application No. 63/407,393, filed on Sep. 16, 2022.

(51) Int. Cl. G16B 30/10; G16B 45/00

(52) U.S. Cl. CPC G16B 30/10; G16B 45/00

(57) ABSTRACT

Systems, methods, and apparatus are described herein for performing sequencing of one or more biological samples in at least two flow cells on a sequencing device. A sequencing system may comprise one or more of a scheduling engine, the sequencing device, and a display. The scheduling engine may maintain scheduling information of a state of compute resources and non-compute resources. The sequencing device may receive the scheduling information from the scheduling engine; determine the state of the compute resources and non-compute resources; determine a sequencing analysis priority associated with performing analysis of the at least two flow cells on the sequencing device; and perform the sequencing task related to the one or more biological samples in the at least two flow cells according to the sequencing analysis priority. The display may display real-time feedback associated with completion of the sequencing task for each flow cell.

### CROSS-REFERENCE TO RELATED APPLICATIONS

This application claims the benefit of U.S. Provisional Patent Application No. 63/407,393, filed Sep. 16, 2022, which is incorporated by reference herein in its entirety.

### BACKGROUND

In recent years, biotechnology firms and research institutions have improved hardware and software platforms to determine a sequence of nucleotide bases (or whole genome) and identify variant calls for nucleotide bases that differ from reference bases of a reference genome. However, sequencing performance of conventional sequencing platforms is generally limited. For example, conventional sequencing platforms may perform sequencing using one flow cell that utilizes a single schedule for completion of sequencing tasks related to the flow cell. As such, the number of samples that can be tested in a given sequencing process is inherently limited. Moreover, typical sequencing methods rely on performing the sequencing process on a large number of samples. However, as the sequencing process performs sequencing via one or more of the same compute or non-compute resources, the sequencing process is performed on a fixed schedule such that a sample is analyzed when the appropriate number of flow cells and the corresponding compute and non-compute resources become available. Moreover, conventional sequencing platforms require a separate sequencing job to be performed for each flow cell that is loaded into the sequencing device in its entirety. Thus, when a sequencing job is stopped prior to completion in favor of a higher-priority job, the analysis that has already been performed is lost and has to be performed again in its entirety at a later time.

### SUMMARY

Systems, methods, and apparatus are described herein for performing sequencing of one or more biological samples in at least two flow cells on a sequencing device. For example, a sequencing device may perform sequencing tasks related to one or more biological samples in at least two flow cells that are loaded into the sequencing device. In some cases, compute resources (e.g., processing resources, data storage resources, memory resources, communication resources, and/or other compute resources) and non-compute resources (e.g., power resources, pumps, heaters, lasers, cameras, and/or portions thereof) may be monitored and controlled on the sequencing device when performing the sequencing tasks. The compute and non-compute resources may be controlled to enable the sequencing tasks to be performed in accordance with one or more sequencing analysis priorities (e.g., prioritizing a makespan of a sequencing task, power consumption of the sequencing task, priority of a flow cell, etc.) that have been identified for the sequencing tasks. The sequencing device may display real-time feedback associated with completion of the sequencing tasks.

In another example, a sequencing system may maintain scheduling information of a state of compute resources and non-compute resources. The sequencing system may receive the scheduling information and determine the state of the compute resources and non-compute resources. In an example, the sequencing system may determine a sequencing analysis priority associated with performing analysis of the two flow cells on the sequencing device. The sequencing system may perform the sequencing task related to the one or more biological samples in the two flow cells according to the sequencing analysis priority. In an example, the sequencing system may perform the sequencing task by controlling at least one compute resource or at least one non-compute resource in accordance with the sequencing analysis priority. In an example, the sequencing system may control the compute resources or non-compute resources based on the state of the compute resources and non-compute resources. In one or more cases, the sequencing system may display a real-time feedback associated with a completion of the sequencing task for each flow cell.

In one or more cases, when the sequencing system performs the sequencing task according to the sequencing analysis priority, the sequencing analysis priority may comprise prioritizing a makespan of the sequencing task, a power consumption for performing the sequencing task, or a priority of a flow cell of the at least two flow cells. In one or more cases, when the sequencing system determines the sequencing analysis priority, the sequencing system determines that a first flow cell is associated with a first priority and a second flow cell is associated with a second priority. In an example, the first priority of the first flow cell has a higher priority than the second priority of the second flow cell. In one or more cases, the sequencing system may perform the sequencing task on the first flow cell and the second flow cell by prioritizing a first set of compute resources and non-compute resources and a second set of compute resources and non-compute resources. In an example, the sequencing system may prioritize the first set of compute resources and non-compute resources to utilize a first amount of power when performing the sequencing task on the first flow cell. In an example, the sequencing system may prioritize the second set of compute resources and non-compute resources to utilize a second amount of power when performing the sequencing task on the second flow cell. In some examples, the first amount of power utilized by the first set of compute resources and non-compute resources may be greater than the second amount of power utilized by the second set of compute resources and non-compute resources. In one or more cases, the sequencing system may perform a first sequencing task on the second flow cell using a first set of compute resources and non-compute resources. The sequencing system may receive an indication to perform a second sequencing task on the first flow cell. Having received the indication, the sequencing system may allocate the first set of compute resources and non-compute resources to performing the second sequencing task. The sequencing system may perform the second sequencing task on the first flow cell using the first set of compute resources and non-compute resources. In one or more cases, the sequencing system may allocate a second set of compute resources and non-compute resources to performing the first sequencing task on the second flow cell. After allocating the second set of compute resources and non-compute resources, the sequencing system may perform the first sequencing task on the second flow cell using the second set of compute resources and non-compute resources.

In one or more cases, the sequencing system may perform a first sequencing task on a second flow cell using a first set of compute resources and non-compute resources. In one or more cases, the sequencing system may receiving an indication to perform a second sequencing task on a first flow cell. In an example, a priority of the second sequencing task performed on the first flow cell may be greater than a priority of the first sequencing task performed on the second flow cell. In some cases, the sequencing system may queue one or more compute resources and non-compute resources of the first set of compute resources and non-compute resources. In an example, the sequencing system may queue the one or more compute resources and non-compute resources to be allocated to performing the second sequencing task upon completion of performing the first sequencing task. After queuing the one or more compute resources and non-compute resources, the sequencing system may allocate the one or more compute resources and non-compute resources of the first set of compute resources and non-compute resources based on the completion the first sequencing task. In one or more cases, the sequencing system may perform the second sequencing task using the allocated one or more compute resources and non-compute resources. In some cases, the sequencing system may save a context of a current state of the first sequencing task. Having saved the context, the sequencing system may allocate the one or more compute resources and non-compute resources of the first set of compute resources and non-compute resources to perform the second sequencing task. In some cases, the sequencing system may perform the second sequencing task on the first flow cell using the allocated one or more compute resources and non-compute resources. In one or more cases, the sequencing system may determine that the second sequencing task is complete. Having determined that the second sequencing task is complete, the sequencing system loads the saved context of the current state of the first sequencing task. In some cases, the sequencing system may allocate the first set of compute resources and non-compute resources to continue performing the first sequencing task. When the first set of compute resources and non-compute resources are allocated, the sequencing system may perform the first sequencing task on the second flow cell based on the saved context of the current state of the first sequencing task. In an example, the first sequencing task and the second sequencing task may comprise a mapping task. In another example, the first sequencing task and the second sequencing task may comprise a sorting task. In another example, the first sequencing task and the second sequencing task may comprise a variant calling task.

In one or more cases, the sequencing system may receive scheduling information from a scheduling table. In an example, the scheduling information comprises the state of the compute resources and non-compute resources and sequencing stages of the one or more biological samples. In one or more cases, the sequencing system may schedule the compute resources and non-compute resources to perform the sequencing task according to the sequencing analysis priority.

### BRIEF DESCRIPTION OF THE DRAWINGS

FIG. 1A illustrates a schematic diagram of a system environment.

FIG. 1B illustrates an example of one or more sequencing subsystems that may be implemented by a sequencing device and/or another computing device for identifying variants or base calls.

FIG. 1C illustrates an example of one or more sequencing subsystems of an example sequencing system.

FIG. 1D is a diagram showing an example of one or more portions of the sequencing device that may comprise compute resources.

FIG. 2 is a flowchart that illustrates a performance of example sequencing of one or more biological samples in at least two flow cells.

FIG. 3 illustrates an example interface displaying a status of example sequencing tasks.

FIG. 4 is a block diagram of an example computing device

### DETAILED DESCRIPTION

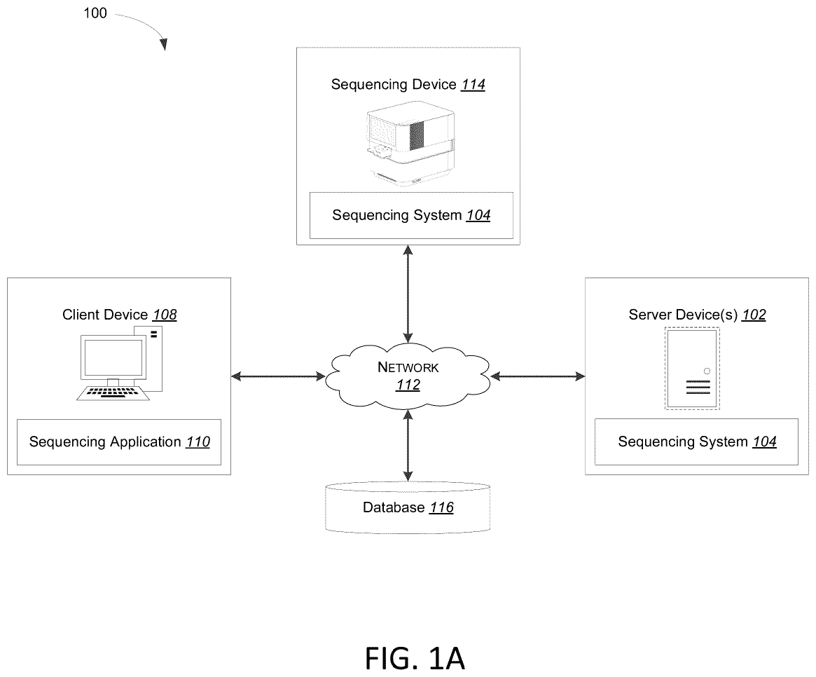

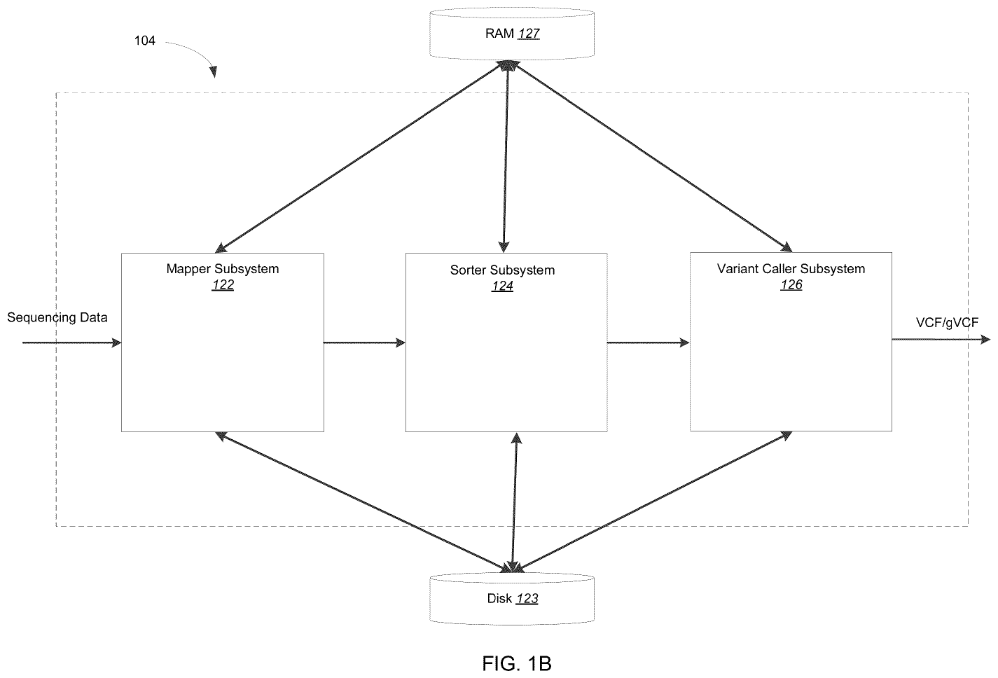

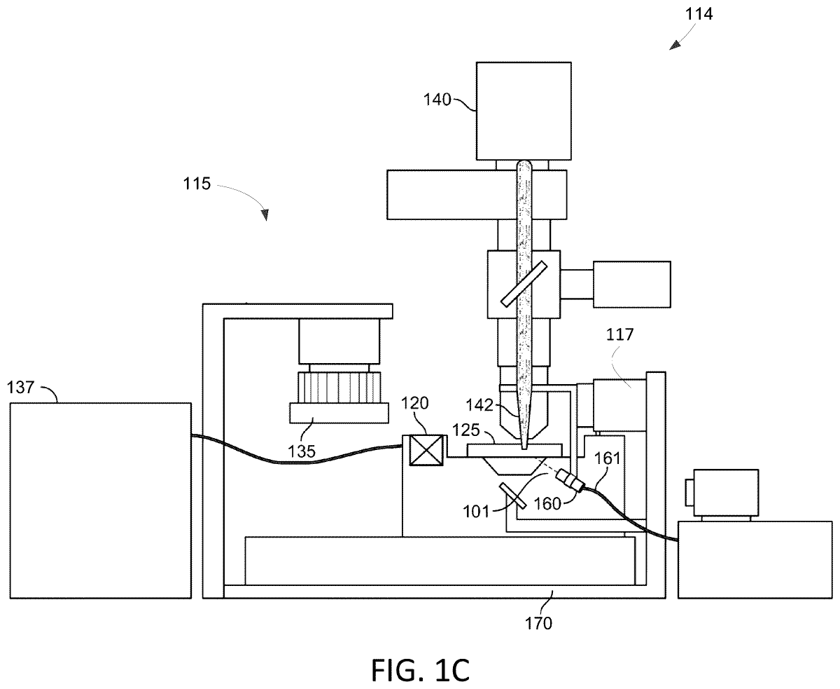

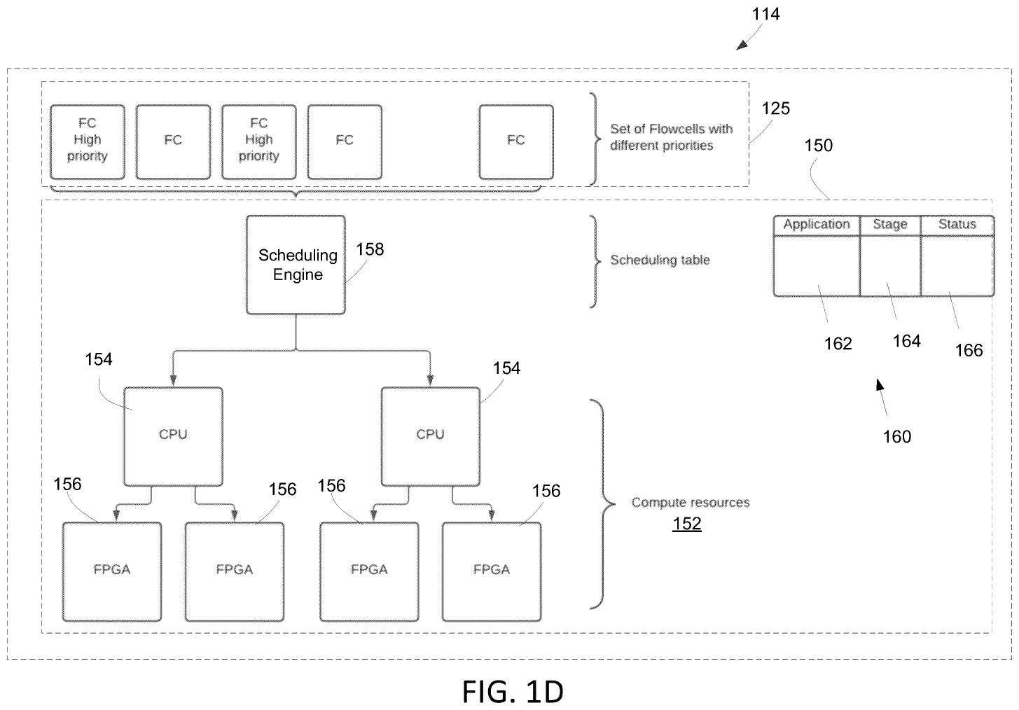

FIG. 1A illustrates a schematic diagram of a system environment (or “environment”) 100, as described herein. As illustrated, the environment 100 includes one or more server device(s) 102 connected to one or more of a client device 108, a database 116, and a sequencing device 114 via a network 112.

As shown in FIG. 1A, the server device(s) 102, the client device 108, the database 116, and the sequencing device 114 may communicate with each other via the network 112. The network 112 may comprise any suitable network over which computing devices can communicate. The network 112 may include a wired and/or wireless communication network. Example wireless communication networks may be comprised of one or more types of radio frequency (RF) communication signals using one or more wireless communication protocols, such as a cellular communication protocol, a wireless local area network (WLAN) or WIFI communication protocol, and/or another wireless communication protocol. Though FIG. 1A illustrates the components of environment 100 communicating via the network 112, it will be appreciated that the components of environment 100 may communicate directly with each other, for example, bypassing the network 112. For example, the client device 108 may communicate directly with the sequencing device 114.

As indicated by FIG. 1A, the sequencing device 114 may comprise a device for sequencing a biological sample. In one or more cases, the biological sample may include, for example, but not limited to, human and non-human deoxyribonucleic acid (DNA) to determine individual nucleotide bases of nucleic-acid sequences (e.g., sequencing by synthesis). In one or more cases, the biological sample may include, for example, but not limited to, human and non-human ribonucleic acid (RNA). The sequencing device 114 may analyze nucleic-acid segments and/or oligonucleotides extracted from samples to generate nucleotide reads and/or other data utilizing computer implemented methods and systems described herein either directly or indirectly on the sequencing device 114. More particularly, the sequencing device 114 may receive and analyze, within nucleotide-sample slides (e.g., flow cells), nucleic-acid sequences extracted from samples. The sequencing device 114 may utilize sequencing-by-synthesis (SBS) to sequence nucleic-acid segments into nucleotide reads.

As further illustrated by FIG. 1A, the server device(s) 102 may generate, receive, analyze, store, and/or transmit digital data, such as data for determining nucleotide-base calls or sequencing nucleic-acid polymers. As shown in FIG. 1A, the sequencing device 114 may generate and send (and the server device(s) 102 may receive) nucleotide reads and/or other data analyzed by the server device(s) 102 for base calling and variant calling.

In one or more cases, the server device(s) 102 may communicate with the client device 108. For example, the server device(s) 102 may send data to the client device 108, including sequencing data or other information, and the server device(s) 102 may receive input from the user via client device 108.

In some cases, the server device(s) 102 may include a distributed collection of servers, in which the server device(s) 102 include a number of server devices distributed across the network 112. In some examples, the distributed server device(s) 102 may be located in the same location or at different physical locations. In other cases, the server device(s) 102 may comprise a content server, an application server, a communication server, a web-hosting server, or another type of server.

In one or more cases, the server device(s) 102 and/or the sequencing device 114 may include a sequencing system 104 or portions thereof. The sequencing system 104 may analyze nucleotide reads and/or other data, such as sequencing metrics generated by the sequencing device 114, to determine nucleotide base sequences for nucleic-acid polymers. For example, the sequencing system 104 may receive raw data generated by the sequencing device 114. The sequencing system 104 may determine a nucleotide base sequence for a nucleic-acid segment, based on the received raw data. In one or more cases, the raw data may be by the sequencing device 114 in a file format, such as, but not limited to, a FASTQ file, that is capable of being recognized for processing. A FASTQ file may include a text file that contains the sequence data from clusters that pass filter on a flow cell. The FASTQ format is a text-based format for storing both a biological sequence (e.g., such as a nucleotide sequence) and corresponding quality scores of the biological sequence. In one or more cases, the sequencing system 104 may process the sequencing data to determine the sequences of nucleotide bases in DNA and/or RNA segments or oligonucleotides.

The sequencing system 104, or one or more portions thereof, residing on the sequencing device 114 may allow for on-device analysis of sequencing data. The sequencing system 104, or one or more portions thereof, residing on the sequencing device 114 may allow the sequencing device to monitor a status of one or more applications operating to perform analysis on sequencing data and/or assist in monitoring a status of resources on the sequencing device 114 to help manage the operation of the sequencing device and portions thereof to accomplish sequencing tasks. As shown in FIG. 1A, the sequencing system 104, or one or more portions thereof, may reside on the server device(s) 102, such that the sequencing device 114 may offload certain sequencing analysis tasks for being performed at the server device(s) 102 and/or request information from the server device(s) 102 to enable the sequencing device 114 to perform as described herein.

FIG. 1B is a diagram showing an example of one or more bioinformatics subsystems that may be implemented by the sequencing system 104, or one or more portions thereof, for performing secondary and/or tertiary forms of sequencing analysis. As shown in FIG. 1B, the sequencing system may implement a mapper subsystem 122, a sorter subsystem 124, and/or a variant caller subsystem 126. Each bioinformatics subsystem may perform a different sequencing task. The mapper subsystem 122 may be implemented to align the reads in sequencing data received from the sequencing device 114 and/or stored at the server device(s) 102. The reads in the sequencing data produced by the sequencing device 114 and/or generated and stored in files may not be included in a single sequence with all DNA information. Instead, the sequencing data produced by the sequencing device 114 may include a number of short subsequences, or reads, with partial DNA information. Read alignment may be performed by the mapper subsystem 122 to map reads to a reference genome and identify the location of each individual read on the reference genome.

After the read alignment is performed at the mapper subsystem 122, the aligned sequencing data may be passed downstream to the sorting subsystem 124 to sort the reads by reference position, and polymerase chain reaction (PCR) or optical duplicates are optionally flagged. An initial sorting phase may be performed by the sorter subsystem 124 on aligned reads returning from the RAM 127. Final sorting and duplicate marking may commence when mapping completes.

The variant caller subsystem 126 may be used to call variants from the aligned and sorted reads in the sequencing data. For example, the variant caller subsystem 126 may receive a sorted file as input and process the reads to generate variant data to be included in a variant call file (VCF) or a genomic variant call format (gVCF) file as output from the variant caller subsystem 126.

Different images may be loaded into a random access memory (RAM) 127 from disk 123 on the sequencing device 114 and/or the server device(s) 102 for each of the subsystems (e.g., mapper subsystem 122, sorter subsystem 124, and variant caller subsystem 126). For example, the RAM 127 may comprise a field programmable gate array (FPGA)-board dynamic RAM (DRAM) on the sequencing device 114 and/or the server device(s) 102 on which different types of data, such as image data, intensity data, voltage data, and/or other types of information associated with genomic sequencing, are loaded from disk 123 for performing different types of analysis using the corresponding subsystem. In one example, the sequencing device 114 may utilize local RAM 127 for loading images of different subsystems to disk and may leverage the server device(s) 102 for storing and/or retrieving from disk 123. The read-mapping process may be performed by FPGA logic on the RAM 127.

In one or more cases, the sequencing system 104 may stay within global memory limits in the RAM 127 and/or on a hard disk drive (HDD) or disk 123 when processing sequencing data. The RAM 127 and the disk 123 may be different types of memory. The RAM 127 may be used to store programs and data that the processor on the sequencing device 114 and/or the server device(s) 102 operating the sequencing system 104, or one or more portions thereof, may use in real-time. The RAM 127 may be volatile and may be erased when the computing device is turned off. The disk 123 may be a permanent storage that is used to store user specific data, programs, and files that may be accessed when the computing device is turned on after being turned off. For example, the sequencing system 104, and/or subsystems thereof, may be stored on disk 123 as computer-executable instructions may be loaded into RAM 127 to operate as described herein. The disk 123 may be a network storage that comprises permanent storage shared on one or more computing devices on a network (e.g., cloud storage system). In addition to the RAM 127, each of the subsystems may have access to memory in the disk 123.

Referring again to FIG. 1A, the client device 108 may generate, store, receive, and/or send digital data for enabling the sequencing processes and analysis described herein. For example, the client device 108 may receive sequencing metrics from the sequencing device 114. The client device 108 may communicate with the server device(s) 102 and/or sequencing device 114 to receive one or more files comprising nucleotide base calls and/or other metrics. In one or more cases, the client device 108 may present or display information pertaining to the nucleotide-base call within a graphical user interface of the client device 108 to a user.

The client device 108 may comprise various types of client devices. In some examples, the client device 108 may be a non-mobile device, such as desktop computer, server, or the like. In other examples, the client device 108 may be a mobile device, such as a laptop, tablet, mobile telephone, smartphone, or the like.

The client device 108 may include a sequencing application 110. The sequencing application 110 may be, for example, a web application or a native application (e.g., a mobile application, desktop application) stored and executed on the client device 108. In one or more cases, the sequencing application 110 may include instructions that (when executed) cause the client device 108 to receive data from the sequencing device 114 and present within a graphical user interface of the client device 108, data, such as, but not limited to data from a variant call file.

The environment 100 may include the database 116. The database 116 may store information such as, but not limited to, variant call files, sample nucleotide sequences, nucleotide reads, nucleotide-base calls, sequencing metrics, population data, and/or other data as described herein. The server device(s) 102, the client device 108, and/or the sequencing device 114 may communicate with the database 116 (e.g., via the network 112) to store and/or access information, such as, but not limited to, variant call files, sample nucleotide sequences, nucleotide reads, nucleotide-base calls, sequencing metrics, population data, and/or other data as described herein.

The environment 100 may be included in a local network or local high-performance computing (HPC) system. In one or more other cases, the environment 100 may be included in a cloud computing environment comprising a plurality of server devices, such as server device(s) 102, having software and/or data distributed thereon. In one or more cases, the sequencing system 104 may be implemented to operate one or more subsystems as described herein. The sequencing system 104 may be distributed across server devices 102 having access to the database 116 via the network 112 in a cloud-based computing system.

The sequencing device 114 may include compute resources and non-compute resources in different sequencing subsystems that may be utilized during operation to enable sequencing analysis. For example, compute resources may include computing hardware and/or software resources, such as computing hardware and/or software for performing analysis of sequencing data, processing, scheduling of non-compute resources, communication via a wired or wireless network, and/or other computing tasks on the sequencing device. The compute resources may include processing resources, data storage resources, memory resources, and/or communication resources. Non-compute resources may include resources that are leveraged for controlling non-computing hardware that may be operated and/or controlled by a computing subsystem for enabling operation of the non-computing hardware on the sequencing device 114. The non-compute resources may include resources on the sequencing device 114 for operating pumps, heaters, lasers, cameras, and/or other non-compute resources on the sequencing device 114. The non-compute resources may include power resources that are used for powering one or more subsystems on the sequencing device 114.

FIG. 1C illustrates an example of one or more sequencing subsystems that may be implemented by the sequencing device 114. The sequencing device 114 may include one or more subsystems that may include non-compute resources. The one or more non-compute resources may be monitored and/or controlled by a computing subsystem when performing analysis on one or more flow cells. In addition to or alternatively, the subsystems may be controlled in response to the schedule/workflow that is determined by the computing subsystem. In one or more cases, the detector subsystem 115 may be configured to perform analysis on one or more flow cells, such as a flow cell 125. For example, the detector subsystem 115 may utilize one or more non-compute resources to perform the analysis on the one or more flow cells. It is noted that FIG. 1C illustrates one flow cell, i.e., flow cell 125, but it should be understood that flow cell 125 may represent multiple flow cells, such as a set of flow cells on which analysis may be performed.

In one or more cases, one or more flow cells 125 may be loaded into the sequencing device 114 and used in the detector subsystem 115. Each flow cell 125 may include a flow cell body having one or more channels that are each configured to convey a solution through the flow cell body. In one or more cases, the flow cell 125 may be configured to be removably coupled to the detector subsystem 115, such that the flow cell 125 may be inserted into the sequencing device 114 and removed from the sequencing device 114. One or more surfaces of the flow cell 125 may be transparent and configured to permit light to pass therethrough. The flow cell body of the flow cell 125 may include fluidic inlet and outlet ports that are in fluid communication with the one or more channels, such that a sample may be included in the respective channels.

The detector subsystem 115 may include non-compute resources, such as, a fluid flow subsystem 137 for directing the flow of reagents (e.g., fluorescent nucleotides, buffers, enzymes, cleavage reagents, etc.) or other solutions to and through a flow cell 125 and waste valve 120. The fluid flow subsystem 137 may include one or more pumps (e.g., EO pumps) that are utilized during operation of the sequencing device 114. The computing subsystem of the sequencing device 114 may control the fluid flow subsystem 137 and/or pumps therein that are responsible for initiating cycles of reactions within one or more flow cells 125. The computing subsystem of the sequencing device 114 may monitor the status (e.g., on/off status, speed, time of use, remaining time of use, etc.) of one or more pumps in the fluid flow system 137 for understanding the status of available non-compute resources that are being utilized and are available. These cycles may be performed with different solutions and/or temperature and flow rates. However, it should be noted that to control the fluid flow subsystem 137 a variety of pumping devices may be operated.

Further, the detector subsystem 115 may include another non-compute resource, such as a heating/cooling element (e.g., heater) of a temperature control subsystem 135, to regulate the reaction conditions within channels of a flow cell 125 and reagent storage areas/containers. The temperature control subsystem 135 may have the heating/cooling element (e.g., heater) positioned underneath the flows cell 125. The heating/cooling element may be configured to heat/cool the flows cell 125 during operation of the detector subsystem 115. The temperature control subsystem 135 may include one or more other heating/cooling elements for other portions of the sequencing device 114. For example, the temperature control subsystem 135 may include heating/cooling elements for controlling the temperature of one or more components, such as driving fans for cooling of the compute components, an illuminator (laser or LED), or other heating/cooling elements for heating/cooling other portions of the sequencing device. The computing subsystem of the sequencing device 114 may monitor the status of the temperature of one or more heating/cooling elements in the temperature control subsystem 135 and/or calculate the amount of time to achieve a given temperature utilizing the one or more heaters in the temperature control subsystem 135 for understanding the available non-compute resources that are being utilized and that are available on the sequencing device 114.

Additionally or alternatively, the detector subsystem 115 may include other non-compute resources, such as, a camera, optics, and/or other components of the camera system 140. Camera system 140 (e.g., a CCD camera) may monitor the flow cell 125 and track the sequencing. In some cases, the camera system 140 may be configured to interact with various filters within a filter switching assembly, lens 142, and focusing laser/focusing laser assembly. A laser device 160 (e.g., an excitation laser within an assembly optionally comprising multiple lasers) may illuminate fluorescent sequencing reactions within the flow cell 125 via laser illumination through, for example, a fiber optic 161. In an example, the laser device 160 may include one or more re-imaging lenses, a fiber optic mounting, and the like. The detector subsystem may also, or alternatively, provide illumination from one or more light emitting diodes (LEDs) through light pipe(s) and lenses. The computing subsystem of the sequencing device 114 may monitor the status of the camera system, the location of the camera system relative to a position in a flow cell (e.g., lanes being analyzed), a usage of the lasers (e.g., power usage and/or duration of time) for understanding the status of an imaging process on one or more flow cells, a usage of the LEDs (e.g., power usage and/or duration of time) and/or status of the LEDs (on/off, intensity, etc.) or another status of the non-compute resources that are being utilized and that are available on the sequencing device 114.

Another non-compute resource in the detector subsystem 115 may be a movable stage 170 upon which the one or more flows cells 125 are placed. The stage 170 may be configured to allow the flows cells 125 to be brought into proper orientation for laser (or other light) excitation 101 of the substrate (e.g., flow cell 125). Further, the stage 170 may be configured to move in relation to a lens 142 and camera system 140, such that the detector subsystem 115 may read different areas of the substrate. It will be appreciated that other components (e.g., the camera, the lens objectives the heater/cooler, etc.) of the detector subsystem 115 may be movable/adjustable to, for example, analyze the one or more flow cells 125. The computing subsystem of the sequencing device 114 may monitor the status of the stage for understanding the status of an imaging process for a flow cell. The computing subsystem of the sequencing device 114 may monitor the status of the stage for understanding the status of non-compute resources that are being utilized and that are available on the sequencing device 114.

Each flow cell, such as flow cell 125, may include its own flow cell subsystem that includes non-compute resources that may be monitored and/or controlled by the computing subsystem of the sequencing device 114 when performing analysis on the one or more flow cells. For example, non-compute resources of the flow cell 125 may include a pump (e.g., an electroosmotic (EO) pump) and/or one or more solutions that may be monitored and/or controlled by the computing subsystem when performing analysis on the sequencing device 114. The pump within each flow cell may induce flow of the solution through the pump and channel between the fluidic inlet and outlet ports. A pump cavity may be provided in the flow cell body. The pump cavity may fluidly communicate with, and be interposed between, an end of the channel and one of the fluidic inlet and outlet ports. In one or more cases, a flow cell, such as flow cell 125, may include contacts that are disposed on at least one of the top and bottom surfaces of the flow cell body. The contacts may be electrically coupled to the pump. The pump may include a porous membrane core that is positioned between electrodes that induce a flow rate of the liquid through the porous core membrane based on a voltage potential maintained between the electrodes. In one or more cases, the flow cell 125 may have clusters of nucleic acid sequences to be sequenced, which may be attached to the substrate of the flow cell 125. The flow cell 125 may include an array of beads, in which each bead may include multiple copies of a single sequence. The computing subsystem of the sequencing device 114 may monitor the status of the pumps and/or the level of solutions for understanding the status of the pumps and/or solution in the flow cell 125.

As discussed herein, the sequencing device 114 may be used for SBS. In SBS, fluorescently labeled modified nucleotides may be used to sequence dense clusters of amplified DNA (possibly millions of clusters) present on the surface of a substrate (e.g., a flow cell). The flow cells containing the nucleic acid samples for sequencing may take the form of arrays of discrete, separately detectable single molecules, arrays of features (or clusters) containing homogeneous populations of particular molecular species, such as amplified nucleic acids having a common sequence, or arrays in which the features are beads comprising molecules of nucleic acid. The nucleic acids may be prepared such that the nucleic acids include an oligonucleotide primer adjacent to an unknown target sequence. To initiate an SBS sequencing cycle, one or more differently labeled nucleotides, and DNA polymerase, etc., can be flowed into/through the flow cell by a fluid flow subsystem, such as fluid flow subsystem 137.

The detector subsystem 115 may include another non-compute resource such as, a reader subsystem that may be used with the camera system 140. The reader subsystem may be configured to aide in analyzing the flow cell 125. For example, when nucleic acid samples have been deposited on the surface of the flow cell 125, the laser device 160 coupled with the optical fiber 161 may be positioned to illuminate the flow cell 125. The flow cell 125 may be placed within a flow cell holder, which may be placed upon movable staging area 170. The flow cell holder may hold the flow cell 125, or a cartridge of one or more flow cells, securely in the proper position or orientation in relation to the laser device 160, the prism (not shown), which directs laser illumination onto the imaging surface, and the camera system 140, while the sequencing occurs. An objective lens component, such as lens 142, may be positioned above the flow cell 125 and capture and monitor the various fluorescent emissions once the fluorophores are illuminated by a laser or other light. In one or more cases, the fluid flow subsystem 137 may direct reagents through the flow cell 125. Alternatively, the objective lens component is positioned below the flow cell 125. The laser device 160 may be similarly positioned or may be adjusted accordingly for the objective lens component to read the fluorescent emissions. In one or more other cases, the flow cell 125 may be viewable from both sides (i.e., top and bottom). As such, the multiple readers or imaging systems may be used to read signals emanating from the channels of the flow cell 125. The flow cell 125 may include one or more complementary metal oxide semiconductor (CMOS) sensors that may be implemented instead of, or in addition to, external cameras and/or optics in the camera system 140. Non-compute resources may be used to drive the capture and read out of the measurements from the CMOS sensor(s). The computing subsystem of the sequencing device 114 may monitor the status of the optical fiber, the laser device 160, LED(s), the CMOS sensors, and/or other non-compute resources for understanding the status of the non-compute resources.

The sequencing device 114 may include an access subsystem configured to move cartridges (e.g., from a receiving position to an engaged position) and/or actuate doors (e.g., to open and close) to provide access to cartridge holders. The cartridges may include one or more flow cells, fluids, reagents, or other materials for being loaded into the sequencing device 114. For example, a cartridge may include one or more rows of flow cells. The cartridge may include multiple flow cells (e.g., 2, 4, 6, or more flow cells) in each row. This may allow for imaging to be performed on multiple different flow cells simultaneously. The flow cells may be of the same or different sizes in a single cartridge. Each cartridge may be loaded with the number of flow cells in the cartridge automatically, or manually. The sequencing device 114 may be loaded automatically, or manually, with one or more cartridges. In one example, the sequencing device 114 may perform analysis on a cartridge and then automatically move the cartridges to allow for analysis on another cartridge. For example, a first cartridge may be in an engaged position for enabling imaging and/or analysis, while a second cartridge may be in a receiving position. After the analysis of the first cartridge, the second cartridge may be moved to the engaged position for enabling imaging and/or analysis. The first cartridge may be moved to the receiving position for unloading of the cartridge and/or loading of another cartridge. Each of the cartridges may be analyzed in a single or multiple sequencing runs. Though examples may be provided herein for performing analysis on one or more flow cells, similar analysis may be performed using cartridges of flow cells, as described herein. The sequencing device 114 may include a status subsystem that includes light bars that provide a visual indication through color changes and/or intensity changes of the status of one or more processes being performed on the sequencing device 114.

Each of the subsystems on the sequencing device may be powered by power resources controlled by a power subsystem 117. The power subsystem 117 may include a power source, such as such as an alternating-current (AC) power source or direct current (DC) power source. The power resources may be non-compute resources controlled by one or more applications for performing different tasks in a sequencing process. The power source may generate a supply voltage for powering the subsystems within the sequencing device 114.

The computing subsystem of the sequencing device 114 may include the compute resources configured to monitor the non-compute resources and/or perform analysis on the one or more flow cells 125 and/or the sequencing data obtained therefrom. FIG. 1D is a diagram showing an example of one or more portions of the sequencing device 114 that may comprise compute resources. As shown in FIG. 1D, the sequencing device 114 may include a computing subsystem 150 comprising compute resources 152. The computing subsystem 150 may be leveraged by the sequencing system 104 for performing sequencing analysis, as described herein. For example, the computing subsystem 150 may comprise one or more processors 154 for performing sequencing analysis, as described herein. In an environment in which the processors 154 are capable of multi-thread processing, the processors 154 may provide multiple threads of execution of within a process for being executed independently and concurrently sharing resources, such as memory resources and/or processing resources, for example. The computing subsystem 150 may include memory located on the sequencing device 114. The memory may comprise RAM and/or disk memory, as described herein. The RAM may comprise one or more FPGAs 156 on the sequencing device 114. As shown in FIG. 1D, each processor 154 may be in communication with one or more FPGAs 156 for performing sequencing analysis, as described herein. Each processor 154 may also have access to disk memory that is located on the sequencing device 114 and/or on a remote computing device, such as one or more server devices 102. Though not shown in FIG. 1D, the computing subsystem 150 may include a communication interface, and/or other computing components configured to operate the sequencing device 114. The communication interface may comprise a transmitter, a receiver, a transceiver, and/or other communication circuits capable of communicating via wired and/or wireless communication interfaces.

The compute resources that may be monitored and/or utilized by the computing subsystem 150 may include, for example, processing resources, data storage resources, memory resources, and/or communication resources. The processing resources may indicate available and/or utilized processing power on one or more processors. The processing resources may indicate a status, time, or speed for processing at one or more processors. The status or time may be a status or time relative to completion of one or more sequencing tasks on the sequencing device 114. In an environment in which the processors 154 are capable of multi-thread processing, the processing resources may be separately monitored for one or more threads. The memory resources may indicate available memory, such as available RAM and/or disk space. The communication resources may include a signal strength or network availability for communicating over a network. For example, the communication resources may indicate a time and/or speed for sending/receiving data over the network. Although the compute and/or non-compute resources may be included in one or more subsystems of the sequencing device 114, it will be appreciated that one or more of the compute and/or non-compute resources may reside locally in one or more subsystems of the sequencing device 114 and one or more other compute and/or non-compute resources may reside remote from the sequencing device 114. For example, the compute resources, memory resources, and/or communication resources may include the resources on the sequencing device 114 and/or one or more server device(s) 102.

The processors 154 on the computing subsystem 150 may load and execute different applications for operating different hardware and/or software portions of the sequencing device 114 when performing one or more sequencing tasks for analyzing the flow cells 125. For example, the processors 154 may load and execute a different application for controlling hardware and/or software for operating different subsystems, or portions thereof, for performing different tasks on the sequencing device 114. Referring again to FIG. 1B, different applications may be loaded into memory and executed on the processors 154 for operating the mapper subsystem 122, the sorter subsystem 124, and/or the variant caller subsystem 126, or portions thereof, for performing secondary or tertiary analysis of sequencing data on the sequencing device 114. Referring to FIG. 1C, different applications may be loaded into memory and executed on the processors 154 for operating the detector subsystem 115, the fluid flow subsystem 137, the temperature control subsystem 135, the flow cell subsystem, the reader subsystem, or portions thereof, for controlling hardware and/or software resources thereon when operating the sequencing device 114.

Referring again to FIG. 1D, the computing subsystem 150 of the sequencing device 114 may comprise a scheduling engine 158 to assist in scheduling tasks and/or workflow for analyzing biological samples of one or more flow cells 125. In another example, the scheduling engine 158 may reside on a remote computing device, such as a server device 102 and/or a client device 104. Each of the flow cells 125 may be loaded into the sequencing device 114 and processed in the sequencing device 114 using the scheduling engine 158 to prioritize sequencing tasks and/or a workflow on the sequencing device 114. The scheduling engine 158 may perform a serial analysis of each of the flow cells 125. For example, the scheduling engine 158 may dedicate compute and/or non-compute resources to each flow cell before allocating compute and/or non-compute resources to another flow cell. The scheduling engine 158 may allocate resources to different flow cells. Each sequencing task may utilize the same or different compute and/or non-compute resources as other sequencing tasks. When a set of compute and/or non-compute resources are scheduled and/or are being utilized for a sequencing task that also needs to be utilized for another sequencing task, the computing subsystem 150 may queue the set of compute resources and/or non-compute resources of to be allocated to performing the subsequent sequencing task upon completion of performing the earlier sequencing task. When different sets of compute and/or non-compute resources are scheduled and/or are utilized for different sequencing tasks, the sequencing tasks may be performed in parallel (e.g., utilizing different FPGAs, processors, etc.) The scheduling engine 158 may be executed via computer-readable instructions and/or machine-readable instructions on one or more processors 154. The scheduling engine 158 may have a comprehensive view of the compute and/or non-compute resources on the sequencing device 114 for performing scheduling and/or workflow management for enabling the sequencing analysis to be performed by the sequencing system 104 for one or more flow cells 125 that may be loaded into the sequencing device 114.

The scheduling of sequencing tasks and/or scheduling of workflows on the sequencing device 114 may take into consideration the current state of compute and/or non-compute resources for processing multiple flow cells 125 that may be loaded into the sequencing device 114. The sequencing device 114 may include a sensor that detects when each of the flow cells 125 is loaded into the sequencing device. For example, each flow cell 125 may include an RFID tag or other device capable of being detected by the sensor (e.g., based on RF signal strength). The scheduling of sequencing tasks and/or a workflow may be updated when a flow cell 125 is loaded into the sequencing device 114 and/or given a priority relative to other flow cells 125 that have been loaded into the sequencing device 114. The sequencing device 114, for example, via the scheduling engine 158, may generate and/or update scheduling of tasks and/or a workflow in response to user input. The priority level of each flow cell 125 may be received by the scheduling engine 158 via a user input on a user interface of the sequencing device 114 and/or via a user interface on a client device 108 and communicated to the sequencing device 114. In another example, one or more flow cells 125 may be indicated as having a priority level or a higher priority level than other flow cells 125 for which a priority level is not indicated. The sequencing device 114 may generate and/or update the scheduling of tasks and/or a workflow automatically. For example, the computing subsystem 150 of the sequencing device 114, via the scheduling engine 158, may schedule tasks and/or a workflow based on a status of one or more compute and/or non-compute resources or other input.

As some sequencing platforms may rely on fixed schedules to perform sequencing tasks and/or without an understanding of the resources that may be utilized in other portions of the system, these sequencing platforms may fail to efficiently utilize the resources on the sequencing device on which the sequencing platform may be operating. As the scheduling engine 158 may include software and/or hardware that provides a comprehensive view of the respective states of compute resources and non-compute resources, the scheduling engine 158 may enable the sequencing device 114 to provide efficient scheduling and utilization of resources for performing analysis for multiple flow cells 125.

The scheduling engine 158 of the computing subsystem 150 of the sequencing device 114 may schedule applications for execution of sequencing tasks in a workflow prepared for multiple flow cells 125 in a sequencing process. The scheduling engine 158 may monitor compute and/or non-compute resources associated with each application being executed by the computing subsystem 150 for controlling hardware and/or software on the sequencing device 114. The scheduling engine 158 may know the applications that are to be executed for performing the tasks on the sequencing device and manage a workflow for analyzing the one or more flow cells 125. The scheduling engine 158 may maintain a job schedule table 160 in memory to assist in scheduling tasks on the sequencing device for analyzing the flow cells 125. The job schedule table 160 may comprise a global view of each application operating on the sequencing device 114 for performing a task. The job schedule table 160 may include job scheduling information that is generated and/or monitored by the scheduling engine 160. The job scheduling information may include one or more applications 162 that have been scheduled and/or are executing on the sequencing device for controlling hardware and/or software for operating one or more portions of the sequencing device 114. The applications 162 may include applications scheduled in a workflow or currently executing for controlling software and/or hardware. The job scheduling information may include a stage 164 that indicates the stage of the workflow for which the application 162 is being executed and/or the stage of the sequencing process at which the corresponding application is being executed for a given flow cell 125 or one or more biological samples. The stage 164 may be comprised of a set of one or more operations that compose a basic unit of work to perform a more complex sequencing task. A stage 164 may be interruptible or non-interruptible. The job scheduling information may include a status 166. The status 166 may include a status of one or more compute and/or non-compute resources being leveraged for the corresponding application. The status 166 may indicate a relative status of the application to completion. The status 166 may indicate the status or amount of compute and/or non-compute resources being utilized by the corresponding application. The status 166 may include a relative state of availability, an amount of resources being utilized, and/or another state of the resources.

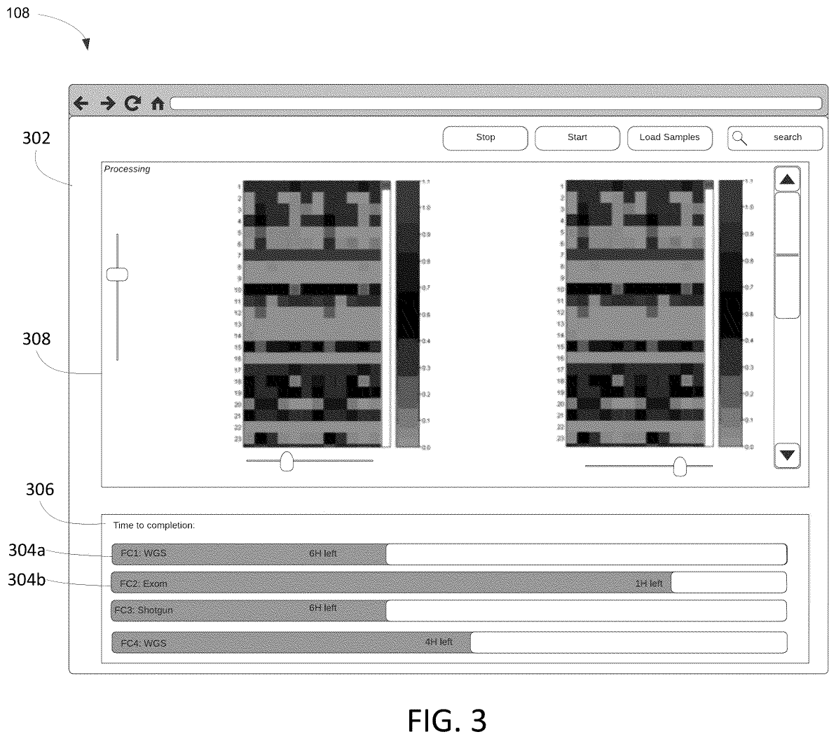

By implementing the scheduling engine 158, the sequencing device 114 may provide efficient scheduling of sequencing tasks, such as, but not limited to, sequencing tasks that include primary analysis, secondary analysis, and/or tertiary analysis on two or more flow cells. Furthermore, the sequencing device 114 may provide real-time feedback, via a graphical user interface (GUI) (e.g., the GUI 302 of client device 108 illustrated in FIG. 3) for estimated time(s) to completion of the one or more sequencing tasks performed by the sequencing device 114.

The computing subsystem 150 of the sequencing device 114, for example, via the scheduling engine 158, may monitor compute resources and/or non-compute resources to inform the scheduling of analysis of multiple flow cells 125 based on one or more sequencing analysis priorities. For example, the scheduling engine 158 may receive a sequencing analysis priority that may indicate one or more flow cells, a make span of one or more tasks or processes, one or more compute resources, and/or one or more non-compute resource to prioritize when analyzing multiple flow cells. The compute and/or non-compute resources on the sequencing device 114 may be divided for performing analysis on multiple flow cells or may be focused for performing analysis on one or more flow cells. The scheduling engine 158 may update the workflow based on different sequencing analysis priorities. Each sequencing analysis priority may indicate to the scheduling engine 158 how to prioritize the compute and/or non-compute resources. For example, the sequencing analysis priority may include one or more flow cells 125 to be given priority over other flow cells 125 that have been loaded into the sequencing device 114. Each flow cell 125 may be given a priority level relative to the priority level of each of the other flow cells 125, such that available compute and/or non-compute resources may be scheduled and/or utilized for processing one or more higher-priority flow cells 125 before scheduling or utilizing compute and/or non-compute resources for processing lower-priority flow cells 125. As described herein, each flow cell may be given priority serially (e.g., as loaded) for imaging and/or analysis, such that each flow cell may be analyzed in the order they are loaded into the system. In another example, each flow cell 125 may be assigned priority level of one or more priority levels, and multiple flow cells may have the same priority level. The scheduling engine 158 may identify the flow cells for being processed in a workflow based on the priority levels. The scheduling engine 158 may schedule available compute resources and/or non-compute resources for being processed according to the priority level of each of the flow cells 125. Where two flow cells 125 have the same priority level assigned to them, the flow cell 125 loaded into the sequencing device 114 earlier in time may be scheduled for utilizing compute and/or non-compute resources before a later loaded flow cell 125.

The scheduling engine 158 may receive a sequencing analysis priority that prioritizes one or more identified compute resources. For example, the sequencing analysis priority may indicate a preference for preserving processing resources, data storage resources, memory resources, communication resources, and/or other compute resources, as described herein. The scheduling engine 158 may have knowledge of the compute resources that are utilized for each application 162 that may be operating at each stage 164 of the sequencing process. The compute resources utilized by a given application may be identified by an average value and/or a high-end value where the compute resources utilized by the application vary. The scheduling engine 158 may generate a workflow and/or update the workflow based on the status of one or more compute resources and/or the indicated preference in the sequencing analysis priority. For example, the sequencing analysis priority may indicate a preference for preserving processing and/or memory resources on the sequencing device 114. The scheduling engine 158 may schedule tasks for processing one or more flow cells in a workflow that preserves the processing and/or memory resources on the sequencing device 114. For example, the tasks may be scheduled such that the processing and/or memory resources are below a predefined level or a level indicated in the received sequencing analysis priority.

The scheduling engine 158 may receive a sequencing analysis priority that prioritizes one or more identified non-compute resources. For example, the sequencing analysis priority may indicate a preference for preserving a power level utilized by the sequencing device 114. The sequencing analysis priority may be received via user input on a user interface of the sequencing device and/or a communication from the sequencing application 110 operating on the client device 108. The scheduling engine 158 may have knowledge of the compute resources and/or non-compute resources that are utilized for each application 162 that may be operating at each stage 164 of the sequencing process. The scheduling engine 158 may have knowledge of how the compute resources and/or non-compute resources that are utilized by a given application affect the non-compute resource indicated as being the sequencing analysis priority. For example, the scheduling engine 158 may have knowledge of the power consumed by each compute resource and/or non-compute resource when executing each application for performing a sequencing task in the sequencing process. The value of the compute resources and/or non-compute resources utilized by each application may be identified by an average value and/or a high-end value where the compute resources and/or non-compute resources utilized by the application vary. The scheduling engine 158 may generate a workflow and/or update the workflow based on the status of one or more compute resources, one or more non-compute resources, and/or the indicated preference in the sequencing analysis priority. The scheduling engine 158 may schedule tasks for processing one or more flow cells in a workflow that preserves the indicated non-compute resources on the sequencing device 114. For example, the tasks may be scheduled such that the power consumption of the sequencing device 118 is below a predefined level or a level indicated in the received sequencing analysis priority. In one example, the heating/cooling element of the temperature control subsystem 135 may be scheduled to start earlier and operate at a lower temperature for a longer period of time to consume a lower amount of power than being operated at a higher temperature for a shorter period of time. In another example, the processing resources and/or memory resources may be limited to limit the load being drawn by the processing resources and/or memory resources.

The scheduling engine 158 may receive a sequencing analysis priority that prioritizes a makespan of a sequencing process or one or more tasks of the sequencing process for one or more flow cells 125. A makespan may include a length of time that elapses from the start of one or more sequencing tasks of a sequencing process for a specific sample or flow cell to the end of the one or more sequencing tasks of the sequencing process for the specific sample or flow cell. The one or more tasks may be performed by one or more applications being monitored by the scheduling engine 158. In one example, the sequencing analysis priority may indicate a preference for prioritizing the makespan of the sequencing process for an identified flow cell 125 (e.g., prioritized flow cell). The scheduling engine 158 may have knowledge of the amount of time to complete each task when different levels of compute resources and/or non-compute resources that are utilized by each application 162 that may be operating at each stage 164 of the sequencing process for performing the task. The scheduling engine 158 may have knowledge of the available compute resources and/or non-compute resources and utilize the available compute and/or non-compute resources for prioritizing the makespan of the sequencing process for an identified flow cell 125. For example, the scheduling engine 158 may maximize the usage of available compute resources and non-compute resources for performing each task in the sequencing process for the identified flow cell. The scheduling engine 158 may generate a workflow and/or update the workflow based on the status of one or more compute resources, one or more non-compute resources, and/or the indicated preference in the sequencing analysis priority. In one example, the heating/cooling element of the temperature control subsystem 135 may be scheduled to start operation at a higher temperature for a shorter period of time to shorten the makespan for the identified flow cell 125. In another example, each of the available processing resources and/or memory resources may be utilized to shorten the makespan by the leveraging the available processing resources and/or memory resources.

The sequencing device 114 may support task migration by utilizing the sequencing engine 158 to update a workflow for shifting compute and/or non-compute resources on the sequencing device 114 from certain tasks that may be performed for one or more flow cells to other tasks that may be performed for one or more other flow cells (e.g., in response to the sequencing analysis priority). For example, if a flow cell is loaded into the sequencing device and/or prioritized over other flow cells or a makespan for processing the flow cell is prioritized, the scheduling engine 158 may shift compute and/or non-compute resources to the applications for performing each task in the workflow for the prioritized flow cell. As different compute and/or non-compute resources may be utilized for performing different tasks in the sequencing process, the scheduling engine 158 may prioritize the use of the compute and non-compute resources for the tasks of the prioritized flow cell. The scheduling engine 158 may understand the amount of compute and/or non-compute resources to be utilized for each task in the sequencing process and may generate a workflow that frees up the compute and/or non-compute resources needed for each task in the sequencing process for the prioritized flow cell. For example, secondary and/or tertiary analysis may utilize a relatively larger amount of the compute and/or non-compute resources (e.g., power resources) on the sequencing device 114 as compared to other task that may be performed on the sequencing device 114. To free up compute and/or non-compute resources for the prioritized flow cell, the scheduling engine 158 may cause the compute and/or non-compute resources utilized by the sequencing device 114 for processing other flow cells to be reduced or to cease being used.

When prioritizing a given flow cell, or cartridge of flow cells, the prioritization may be given for loading and/or analyzing the flow cells. For example, a flow cell, or cartridge of flow cells, may be prioritized at loading to prioritize the flow cell, or cartridge of flow cells, once the compute and/or non-compute resources become available to allow the analysis that is currently being performed to complete. The prioritized flow cells, or cartridge of flow cells, may be moved to a location for imaging and/or analysis ahead of other flow cells. In another example, a flow cell, or cartridge of flow cells, may be given priority for analysis, such that analysis that is currently being performed may be stopped, or stopped at a predefined point in the analysis, and the compute and/or non-compute resources may be utilized for the prioritized flow cell, or cartridge of flow cells. When analysis is stopped, the current results may be stored (e.g., spilled to disk) for being uploaded when the analysis resumes.

The sequencing device 114 (e.g., via the scheduling engine 158) may support task migration by allowing preemption of the task that is currently being performed on a lower-priority flow cell by one or more tasks for processing the prioritized flow cell. In order to preserve the analysis that has been performed as a result of completion of the tasks on the lower-priority flow cell, the sequencing device may store a context of one or more tasks that have been performed and/or that are currently being performed for processing the lower-priority flow cell. The context may include a hardware and/or software context. The scheduling engine 158 may allow the sequencing task that is currently being performed for the lower-priority flow cell to complete before performing preemption and/or storing the context of the task. When the sequencing device 114 allows the current task associated with a flow cell to finish, the scheduling engine 158 may prepare a workflow for shifting compute and/or non-compute resources to the sequencing process for another flow cell. The scheduling engine 158 may perform preemption of the task and/or store the context of the task prior to its completion. The context may be stored to disk in memory locally at the sequencing device 114 and/or on a remote device, such as a server device for example. The context may include a current state of the sequencing task that is being performed and/or the state of compute resources (e.g., memory resources and/or processing resources, including resources at one or more CPUs and/or FPGAs) before preemption. For example, the current state of the sequencing task may include information corresponding to the current progress of the sequencing task, the type and/or number of compute resources and non-compute resources being utilized, the current status of the compute resources and non-compute resources being utilized, the subsystem(s) and/or applications(s) for performing the current sequencing task, the stage of the workflow for which the application is being executed for performing the task, the stage of the sequencing process at which the corresponding application is being executed, and/or other context information that would enable the sequencing device 114 to resume performing the sequencing task for the lower-priority flow cell at a later time. In one example, if the sequencing device 114 is performing secondary analysis of the sequencing data generated on the sequencing device 114, the context that is stored may identify the bioinformatics subsystem (e.g., the mapper subsystem 122, the sorter subsystem 124, and/or the variant caller subsystem 126) or application that is performing the secondary analysis and/or the portion of the sequencing data that has been processed by the bioinformatics subsystem and/or application. The results of the analysis that has been performed may also be stored in the context.

The context of one or more sequencing tasks may be stored on disk at the sequencing device 114 and/or may be transmitted to a remote computing device, such as a server device 102. Having saved the context of the current state of the sequencing task for the lower-priority flow cell, the sequencing device 114 may allocate the compute resources and non-compute resources and load the one or more applications performing the sequencing tasks for the higher-priority flow cell using the allocated compute resources and non-compute resources. The processors 154 of the sequencing device 114 may continue to load the applications for performing the sequencing tasks according to the workflow generated by the scheduling engine 158 for completing the sequencing process for the higher-priority flow cell. After the sequencing process is completed for the higher-priority flow cell, the sequencing device 114 may load the saved context for the other flow cell (e.g., including the application(s) for performing the sequencing task). Preemption may occur multiple times and the context may be determined and/or saved multiple times (e.g., for each preemption). The scheduling engine 158 of the sequencing device 114 may subsequently allocate one or more compute resources and/or non-compute resources to resume performing the sequencing task.

The sequencing device 114 may enable a partial reconfiguration of one or more FPGAs 156 when performing task preemption. As described herein, different software and/or images may be loaded to the FPGAs 156 for operating different portions of the bioinformatics subsystem (e.g., the mapper subsystem 122, the sorter subsystem 124, and/or the variant caller subsystem 126) depending on the state of the tasks being performed. When one of the portions of the bioinformatics subsystem is being utilized for performing analysis on a flow cell that is preempted for performing analysis using another portion of the bioinformatics subsystem, the software and/or images for operating another portion of the bioinformatics subsystem may be loaded into the one or more FPGAs 156.

If the sequencing device 114 is processing one or more flow cells 125, the applications that are being operated for performing one or more tasks in the sequencing process for one or more flow cells 125 may be preempted for prioritizing one or more tasks in the sequencing process for one or more flow cells 125. This preemption may be performed in response to one or more flow cells 125 being loaded into the sequencing device and/or receipt of a sequencing analysis priority (e.g., different sequencing analysis priority). For example, the scheduling engine 158 may identify another flow cell 125 that is loaded into the sequencing device and/or a change in a sequencing analysis priority and update the workflow for processing the flow cells 125.

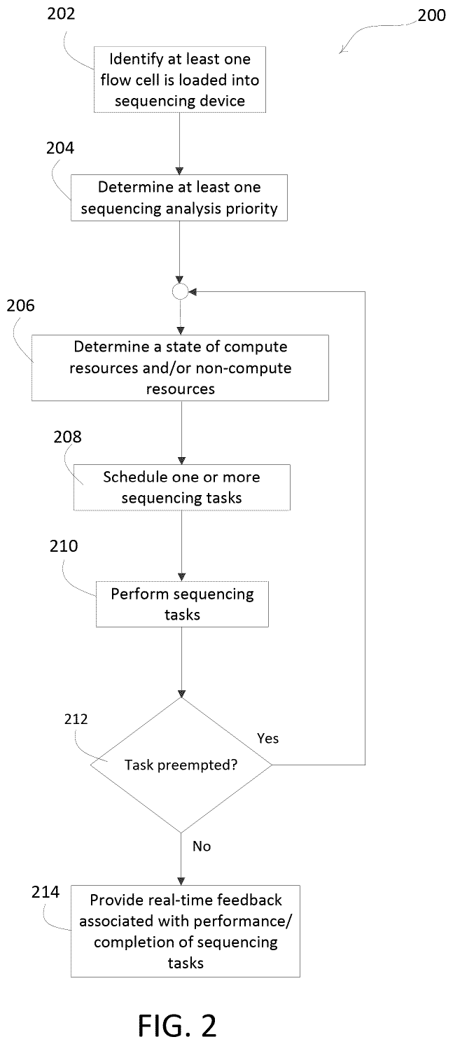

FIG. 2 is a flowchart that illustrates a procedure 200 for performing one or more sequencing tasks of a sequencing process for one or more biological samples of at least two flow cells. The one or more portions of the procedure 200 may be performed by one or more computing devices. For example, the one or more portions of the procedure 200 may be performed by one or more sequencing devices. One or more portions of the procedure 200 may be stored in memory as computer-readable or machine-readable instructions that may be executed by a processor of the one or more computing devices. One or more portions of the procedure 200 may be performed by the scheduling engine and/or one or more subsystems operating on the sequencing device. Though portions of the procedure 200 may be described herein as being performed by a sequencing device, the procedure 200, or portions thereof, may be performed by another computing device or distributed across multiple computing devices, such as one or more sequencing devices, one or more client devices, and/or one or more server devices.

The procedure 200 may begin at 202. As shown in FIG. 2, at 202 the sequencing device 114 may identify at least one flow cell is loaded into the sequencing device 114. For example, a user may load a first flow cell (e.g., FC1) into the detector subsystem 115 of the sequencing device 114. The sequencing device may include a sensor that detects when the flow cell is loaded into the sequencing device. For example, the flow cell may include an RFID tag or other device capable of being detected by the sensor. In another example, having received the first flow cell FC1 on the sequencing device 114, the sequencing device 114 may identify that a second flow cell (e.g., FC2) has been loaded into the sequencing device 114. In one or more cases, having received the flow cell, the sequencing device 114 may also determine a sequencing task associated with the flow cell.

The sequencing device 114 may determine at least one sequencing analysis priority at 204. For example, the scheduling engine 158 of the sequencing device 114 may determine at least one sequencing analysis priority for scheduling tasks and/or a workflow for assigning compute and/or non-compute resources on the sequencing device 114. The sequencing device 114 may determine the sequencing analysis priority associated with performing analysis of the at least two flow cells on the sequencing device. For example, having identified the second flow cell FC2 as being loaded in the sequencing device 114, the sequencing device 114 may determine that the first flow cell FC1 is associated with a first sequencing analysis priority and that the second flow cell FC2 is associated with a second sequencing analysis priority. A sequencing analysis priority may comprise a value, score, or other like indicators that can provide a ranking or relative priority level. The sequencing analysis priority may be associated with a priority for assigning one or more respective compute and/or non-compute resources for performing sequencing tasks for the prioritized flow cell. The sequencing analysis priority may be associated with a priority for scheduling sequencing tasks for one or more prioritized flow cells. For example, the first sequencing analysis priority may prioritize a makespan for performing a sequencing process of the first flow cell FC1. In another example, the sequencing analysis priority may merely identify the first flow cell FC1 as a prioritized flow cell or a flow cell with a higher priority than the second flow cell FC2. Though a sequencing analysis priority is described for prioritizing at least one identified flow cell, the sequencing analysis priority may include other priorities for consideration by the sequencing device when scheduling tasks and/or allocating compute and/or non-compute resources for a workflow. For example, the sequencing priority may indicate one or more compute resources (e.g., processing resources, data storage resources, memory resources, communication resources, and/or other resources) and/or non-compute resources (e.g., power resources and/or other non-compute resources on the sequencing device). Additionally, although a single sequencing priority is used as an example herein, one or more sequencing priorities may be considered when scheduling tasks and/or allocating compute and/or non-compute resources for a workflow. The sequencing analysis priority may be input into a user interface of the sequencing device 114 by a user or may be received from another device, such as a client device 108. The sequencing device 114 may not determine a sequencing analysis priority for the first flow cell FC1 for the cases in which the first flow cell FC1 is the only flow cell loaded into the sequencing device 114.

A state of the compute resources and/or non-compute resources may be determined at 206 by the sequencing device 114. For example, the current state of the compute resources and/or non-compute resources may be determined by the scheduling engine 158 of the sequencing device 114 for scheduling tasks and/or a workflow for assigning the compute resources and/or non-compute resources on the sequencing device 114. In one example, upon receiving the first flow cell FC1, the sequencing device 114 and sequencing system 104 may utilize one or more of the compute resources (e.g., one or more CPUs and one or more FPGAs) and/or one or more of the non-compute resources (e.g., fluid flow subsystem 137 and temperature control subsystem 135), as described herein, for performing one or more tasks in a sequencing process for the first flow cell FC1. The sequencing device 114 may determine a state of the available compute resources and non-compute resources on the sequencing device for assigning the compute and/or non-compute resources for sequencing tasks related to the analysis of the first flow cell FC1 and/or the second flow cell FC2.

At 208, the sequencing device 114 may schedule one or more sequencing tasks for being performed on the sequencing device 114. For example, the scheduling engine 158 may schedule the one or more sequencing tasks, such as in a workflow, based on the at least one sequencing analysis priority and/or the current state of available compute and/or non-compute resources. In the example provided herein, the scheduling engine 158 may schedule the one or more sequencing tasks and prioritize the allocation of the compute and/or non-compute resources for enabling performance of a sequencing process for the first flow cell FC1. The one or more tasks that are scheduled may also include one or more tasks for enabling performance of a sequencing process for the second flow cell FC1. One or more sequencing tasks may be performed by an application and/or subsystem residing on the sequencing device 114. The scheduling engine 158 may maintain a job scheduling table 160 that includes the schedule of the applications executing the one or more tasks (or merely maintains the one or more tasks), a stage, and/or a status associated with the application. In the example provided above, the scheduling engine 158 may prioritize the sequencing tasks for being performed for the first flow cell FC1 and may prioritize the allocation of available compute and non-compute resources for the first flow cell FC1.

The scheduled sequencing tasks may be performed at 210 by the sequencing device 114. For example, the sequencing device 114 may perform the sequencing tasks related to the one or more biological samples in the respective flow cells according to the scheduled workflow. For instance, for the cases in which the first flow cell FC1 has a higher sequencing analysis priority than the sequencing analysis priority of the second flow cell FC2, the sequencing device 114 may prioritize the allocation of compute and/or non-compute resources for performing the sequencing tasks related to the one or more biological samples in the first flow cell FC1. In such cases, the sequencing device 114 may queue the sequencing task related to the second flow cell FC2 until one or more compute resources and non-compute resources become available. The sequencing device 114 may be configured to perform the sequencing tasks by controlling at least one compute resource and/or at least one non-compute resource.

In response to determining that the compute resources and/or non-compute resources are available to perform the queued sequencing tasks related to the second flow cell FC2, the sequencing device 114 may allocate the available compute resources and non-compute resources and perform the sequencing task related to the one or more biological samples in the second flow cell FC2. It is noted that such queuing mechanisms may allow users to maximize use of compute resources and non-compute resources, even if the user is not on-site or unavailable to access the sequencing device 114.

While performing one or more sequencing tasks, the sequencing device 114 may determine that the one or more sequencing tasks have been preempted at 212. The preemption may be based on at least one additional sequencing analysis priority. For example, the sequencing device may identify that a third flow cell FC3 has been loaded into the sequencing device 114 and/or assigned a higher priority than the flow cell for which the sequencing tasks are currently being performed on the sequencing device. In another example, the sequencing analysis priority may indicate a priority for reducing power usage on the sequencing device, or reducing a makespan of the first flow cell FC1 or the second flow cell FC2. In another example, the sequencing analysis priority may indicate a change in relative priority between the first flow cell FC1 and the second flow cell FC2. If the current task is preempted at 212 (e.g., based on a subsequently received sequencing analysis priority), the sequencing device 114 may determine the current state of the compute and/or non-compute resources and update the workflow for scheduling one or more sequencing tasks (e.g., according to the subsequently received sequencing analysis priority).

In an example in which the schedule of the one or more sequencing tasks causes a change in the current sequencing task (e.g., due to prioritization of another flow cell, such as a third flow cell FC3 that has been loaded into the sequencing device 114 or a change in priority between the first flow cell FC1 and the second flow cell FC2), the sequencing device may adjust the allocation of compute and/or non-compute resources for performing the sequencing tasks in the updated workflow. Before the second sequencing task preempts the first sequencing task, the sequencing device 114 may save a context of a current state of the current sequencing task being performed on the first flow cell FC1 to enable the sequencing device 114 to resume performing the sequencing task at a later time. Having saved the context of the current state of the first sequencing task, the sequencing device 114 may allocate the compute resources and/or non-compute resources and perform the newly scheduled sequencing task using the allocated compute resources and/or non-compute resources. The scheduling engine 158 may include the loading of the stored context at the appropriate location in the workflow (e.g., after completion of one or more tasks of the prioritized flow cells and/or identification of available compute and/or non-compute resources) for resuming performance of the sequencing tasks that were previously preempted.

The sequencing device 114 may provide real-time feedback associated with the performance and/or completion of sequencing tasks is provided at 210. For example, the sequencing device 114 may provide real-time feedback indicating the current status and/or completion of the sequencing tasks to the sequencing application 110 implemented on the client device 108 and/or on a user interface operating locally on the sequencing device 114.

As illustrated in FIG. 3, the sequencing application 110 and/or a local application operating on the sequencing device 114 may display a GUI 302 that depicts a real-time feedback associated with a completion of a sequencing task for one or more flow cells. For example, GUI 302 displays a processing window 308 to provide a graphical representation of a sequencing analysis that is performed on one or more biological samples associated with a respective flow cell. The GUI 302 displays a sequencing analysis status window 306. The information in the sequencing analysis status window may be determined and/or generated from the information in the job scheduling table 160 and/or other information monitored by the scheduling engine 158. Status window 306 may provide indicators, such as indicators 304a and 304b, to indicate a time to completion. For example, indicator 304a may indicate that a time to complete the sequencing task for the first flow cell FC1 is six hours, though other indications may be provided. Indicator 304b may indicate that a time to complete the sequencing task for the second flow cell FC2 is one hour, though other indications may be provided. The indicators, such as indicators 304a and 304b, may dynamically change based on the progress of the associated sequencing task and/or the compute/non-compute resources available or scheduled to be available over a period of time. For example, as the sequencing task progresses, an area of the indicator may be illuminated a distinct color. The illuminated area may increase as the sequencing task progresses and may decrease if the sequencing task regresses. It is noted that the GUI 302 is displayed on the client device 108 via the sequencing application 110. However, it should be appreciated that the GUI 302 may be displayed on other devices, such as a display associated with the server device(s) 102 or a display associated with the sequencing device 114 (e.g., a display that is integrated with the sequencing device 114).

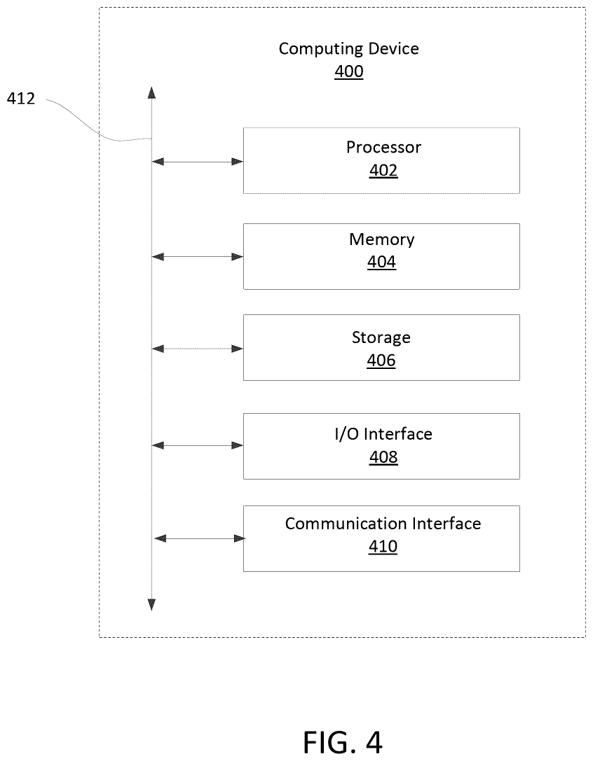

FIG. 4 is a block diagram illustrating an example computing device 400. One or more computing devices such as the computing device 400 may implement one or more features for generating and/or processing sequencing tasks, as described herein. For example, the computing device 400 may comprise one or more of the sequencing device 114, the client device 108, and/or the server device(s) 102 shown in FIG. 1A. As shown by FIG. 4, the computing device 400 may comprise a processor 402, a memory 404, a storage device 406, an I/O interface 408, and/or a communication interface 410, which may be communicatively coupled by way of a communication infrastructure 412. It should be appreciated that the computing device 400 may include fewer or more components than those shown in FIG. 4.

The processor 402 may include hardware for executing instructions, such as those making up a computer program. In examples, to execute instructions for dynamically modifying workflows, the processor 402 may retrieve (or fetch) the instructions from an internal register, an internal cache, the memory 404, or the storage device 406 and decode and execute the instructions. The memory 404 may be a volatile or non-volatile memory used for storing data, metadata, computer-readable or machine-readable instructions, and/or programs for execution by the processor(s) for operating as described herein. The storage device 406 may include storage, such as a hard disk, flash disk drive, or other digital storage device, for storing data or instructions for performing the methods described herein.

The I/O interface 408 may allow a user to provide input to, receive output from, and/or otherwise transfer data to and receive data from the computing device 400. The I/O interface 408 may include a mouse, a keypad or a keyboard, a touch screen, a camera, an optical scanner, network interface, modem, other known I/O devices or a combination of such I/O interfaces. The I/O interface 408 may include one or more devices for presenting output to a user, including, but not limited to, a graphics engine, a display (e.g., a display screen), one or more output drivers (e.g., display drivers), one or more audio speakers, and one or more audio drivers. The I/O interface 408 may be configured to provide graphical data to a display for presentation to a user. The graphical data may be representative of one or more graphical user interfaces and/or any other graphical content.

The communication interface 410 may include hardware, software, or both. In any event, the communication interface 410 may provide one or more interfaces for communication (such as, for example, packet-based communication) between the computing device 400 and one or more other computing devices or networks. The communication may be a wired or wireless communication. As an example, and not by way of limitation, the communication interface 410 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 410 may facilitate communications with various types of wired or wireless networks. The communication interface 410 may also facilitate communications using various communication protocols. The communication infrastructure 412 may also include hardware, software, or both that couples components of the computing device 400 to each other. For example, the communication interface 410 may use one or more networks and/or protocols to enable a plurality of computing devices connected by a particular infrastructure to communicate with each other to perform one or more aspects of the processes described herein. To illustrate, the sequencing process may allow a plurality of devices (e.g., a client device, sequencing device, and server device(s)) to exchange information such as sequencing data and error notifications.

In addition to what has been described herein, the methods and systems may also be implemented in a computer program(s), software, or firmware incorporated in one or more computer-readable media for execution by a computer(s) or processor(s), for example. Examples of computer-readable media include electronic signals (transmitted over wired or wireless connections) and tangible/non-transitory computer-readable storage media. Examples of tangible/non-transitory computer-readable storage media include, but are not limited to, a read only memory (ROM), a random-access memory (RAM), removable disks, and optical media such as CD-ROM disks, and digital versatile disks (DVDs).

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure.

What is claimed is:

1. A computer-implemented method for performing sequencing of one or more biological samples in at least two flow cells on a sequencing device, the method comprising:
analyzing compute resources and non-compute resources associated with the sequencing device to determine a state of the compute resources and non-compute resources;
determining a sequencing analysis priority associated with performing analysis of the at least two flow cells on the sequencing device;
performing a sequencing task related to the one or more biological samples in at least one of the at least two flow cells according to the sequencing analysis priority, wherein the sequencing task is performed by controlling at least one compute resource or at least one non-compute resource in accordance with the sequencing analysis priority, and wherein the at least one compute resource or the at least one non-compute resource is controlled based on the state of the compute resources and non-compute resources; and
displaying real-time feedback associated with completion of the sequencing task for each flow cell on a display.

2. The method of claim 1, wherein the sequencing analysis priority comprises prioritizing a makespan of a sequencing process comprising the sequencing task, a power consumption for performing the sequencing task, or a priority of a flow cell of the at least two flow cells.

3. The method of claim 1, wherein determining the sequencing analysis priority further comprising determining that a first flow cell is associated with a first priority and a second flow cell is associated with a second priority, wherein the first priority is a relatively higher priority than the second priority.

4. The method of claim 3, further comprising performing the sequencing task on the first flow cell and the second flow cell by prioritizing a first set of compute resources and non-compute resources to utilize a first amount of power when performing the sequencing task on the first flow cell and a second set of compute resources and non-compute resources to utilize a second amount of power when performing the sequencing task on the second flow cell.

5. The method of claim 4, wherein the first amount of power is greater than the second amount of power.

6. The method of claim 3, further comprising:
performing a first sequencing task on the second flow cell using a first set of compute resources and non-compute resources;
receiving an indication to perform a second sequencing task on the first flow cell;
allocating the first set of compute resources and non-compute resources to performing the second sequencing task; and
performing the second sequencing task on the first flow cell using the first set of compute resources and non-compute resources.

7. The method of claim 6, further comprising:
allocating a second set of compute resources and non-compute resources to performing the first sequencing task on the second flow cell; and
performing the first sequencing task on the second flow cell using the second set of compute resources and non-compute resources.

8. The method of claim 1, further comprising:
performing a first sequencing task on a second flow cell using a first set of compute resources and non-compute resources; and
receiving an indication to perform a second sequencing task on a first flow cell, wherein a priority of the second sequencing task is greater than a priority of the first sequencing task.

9. The method of claim 8, further comprising:
queuing one or more compute resources and non-compute resources of the first set of compute resources and non-compute resources to be allocated to performing the second sequencing task upon completion of performing the first sequencing task;
allocating the one or more compute resources and non-compute resources of the first set of compute resources and non-compute resources based on the completion the first sequencing task; and
performing the second sequencing task using the allocated one or more compute resources and non-compute resources.

10. The method of claim 8, further comprising:
saving a context of a current state of the first sequencing task;
allocating the one or more compute resources and non-compute resources of the first set of compute resources and non-compute resources to perform the second sequencing task; and
performing the second sequencing task on the first flow cell using the allocated one or more compute resources and non-compute resources.

11. The method of claim 10, further comprising:
determining the second sequencing task is complete;
loading the saved context of the current state of the first sequencing task;
allocating the first set of compute resources and non-compute resources to continue performing the first sequencing task; and
performing the first sequencing task on the second flow cell based on the saved context of the current state of the first sequencing task.

12. The method of claim 11, wherein the first sequencing task and the second sequencing task comprise one of a mapping task, a sorting task, or a variant calling task.

13. The method of claim 1, further comprising:
receiving scheduling information from a scheduling table, wherein the scheduling information comprises the state of the compute resources and non-compute resources and sequencing stages of the one or more biological samples; and
scheduling the compute resources and non-compute resources to perform the sequencing task based on the scheduling information.

14. A sequencing system for performing sequencing of one or more biological samples in at least two flow cells on a sequencing device, the system comprising:
a scheduling engine configured to maintain scheduling information of a state of compute resources and non-compute resources;
the compute resources and the non-compute resources configured to perform a sequencing task;
the sequencing device configured to:
receive the scheduling information from the scheduling engine;
determine the state of the compute resources and non-compute resources;
determine a sequencing analysis priority associated with performing analysis of the at least two flow cells on the sequencing device; and
perform the sequencing task related to the one or more biological samples in the at least two flow cells according to the sequencing analysis priority, wherein the sequencing task is performed by controlling at least one compute resource or at least one non-compute resource in accordance with the sequencing analysis priority, and wherein the at least one compute resource or the at least one non-compute resource is controlled based on the state of the compute resources and non-compute resources; and
a display configured to display real-time feedback associated with completion of the sequencing task for each flow cell.

15. The sequencing system of claim 14, wherein the sequencing analysis priority comprises a prioritization of a makespan of a sequencing process comprising the sequencing task, a power consumption for performing the sequencing task, or a priority of a flow cell of the at least two flow cells.

16. The sequencing system of claim 14, wherein the sequencing device is further configured to determine the sequencing analysis priority by determining that a first flow cell is associated with a first priority and a second flow cell is associated with a second priority, wherein the first priority is a higher priority than the second priority.

17. The sequencing system of claim 16, wherein the sequencing device is further configured to:
perform a first sequencing task on the second flow cell using a first set of compute resources and non-compute resources;
receive an indication to perform a second sequencing task on the first flow cell;
allocate the first set of compute resources and non-compute resources to performing the second sequencing task; and
perform the second sequencing task on the first flow cell using the first set of compute resources and non-compute resources.

18. The sequencing system of claim 14, wherein the sequencing device is further configured to:
perform a first sequencing task on a second flow cell using a first set of compute resources and non-compute resources; and
receive an indication to perform a second sequencing task on a first flow cell, wherein a priority of the second sequencing task is greater than a priority of the first sequencing task.

19. The sequencing system of claim 18, wherein the sequencing device is further configured to:
save a context of a current state of the first sequencing task;
allocate the one or more compute resources and non-compute resources of the first set of compute resources and non-compute resources to perform the second sequencing task; and
perform the second sequencing task on the first flow cell using the allocated one or more compute resources and non-compute resources.

20. The sequencing system of claim 19, wherein the sequencing device is further configured to:
determine the second sequencing task is complete;
load the saved context of the current state of the first sequencing task;
allocate the first set of compute resources and non-compute resources to continue performing the first sequencing task; and
perform the first sequencing task on the second flow cell based on the saved context of the current state of the first sequencing task.
